(19) United States
(12) Patent Application Publication
(10) Pub. No.: US 2015/0045739 A1
(43) Pub. Date: Feb. 12, 2015

(54) METHOD FOR TREATING OTIC INFECTIONS AFTER TYMPANOSTOMY TUBE PLACEMENT

(71) Applicant: NOVARTIS AG, Basel (CH)

(72) Inventors: Wolf-Ulrich Nickel, Hudson Oaks, TX (US); Peter S. Roland, Irving, TX (US); Firoz G. Vohra, Flower Mound, TX (US)

(21) Appl. No.: 14/458,176

(22) Filed: Aug. 12, 2014

Related U.S. Application Data
(60) Provisional application No. 61/864,713, filed on Aug. 12, 2013.

(51) Int. Cl. A61M 5/32; A61K 45/06; A61K 31/5383; A61F 11/00; A61M 5/00

(52) U.S. Cl. CPC A61M 5/3286; A61F 11/00; A61M 5/002; A61K 31/5383; A61K 45/06; A61F 11/002
USPC 604/218; 514/230.5

(57) ABSTRACT

The present invention relates to methods for treating a microbial infection comprising administering a composition comprising one or more antibiotic compounds to the site of the infection by instilling the composition into the tympanostomy tube. A delivery cannula can be used to instill the composition into the tympanostomy tube.

### CROSS-REFERENCE TO RELATED APPLICATION

This application claims priority under 35 U.S.C. §119 to U.S. Provisional Patent Application No. 61/864,713, filed Aug. 12, 2013 the entire contents of which are incorporated herein by reference.

### TECHNICAL FIELD OF THE INVENTION

The present invention generally relates to methods for treating otic infections, and specifically relates to the treatment of otic infections such as acute otitis media with tympanostomy tubes.

### BACKGROUND OF THE INVENTION

Tympanostomy tubes are small cylinders inserted in the tympanic membrane of the ear, usually in response to persistent ear infections and fluid buildup behind the membrane. The fluid buildup and pressure created by such infections is extremely uncomfortable for the patient, who is typically a child or young adult. The tympanostomy tube allows such fluid to drain and equalizes pressure behind the tympanic membrane, relieving discomfort for the patient.

Unfortunately, middle ear infections can continue or reoccur after tympanostomy tubes are inserted. Pathogens associated with chronic or recurrent middle ear infections after tympanostomy tube insertion include Streptococcus pneumonia, Staphylococcus aureus, Haemophilus influenza, Moraxella catarrhalis, and Streptococcus pyogenes. These infections are currently treated using antibiotics, either alone or in combination with an anti-inflammatory drug such as a steroid. The current standard of care prescribes antibiotics applied topically to the ear, with a typical administration regimen requiring that 4 or more drops of an antibiotic formulation be instilled into the ear twice a day.

Unfortunately, the standard of care regimen can be quite difficult to comply with for the patient (and for the patient's caregiver, in the frequent case of pediatric infection). The quantity of drops actually entering the ear can be difficult to verify, and therefore a seven-day course of administration is usually recommended. Because of the difficulty in maintaining compliance, particularly in the context of pediatric infections, improved methods for treating middle ear infections following tympanostomy tube insertion are needed.

### BRIEF SUMMARY OF THE INVENTION

The present invention relates in one aspect to methods for the treatment of ear infections following the placement of a tympanostomy tube. Such infections are typically infections of the middle ear space and are characterized by otorrhea (discharge) seeping from the newly inserted tympanostomy tube (otitis media at the time of tube placement or “OMTT”) or from the in-place tympanostomy tube (acute otitis media with tympanostomy tubes or “AOMT”). The methods of the present invention involve the administration of a composition comprising one or more antibiotic compounds, alone or in combination with one or more anti-inflammatory compounds.

In one embodiment of the present invention, a composition comprising an antibiotic is administered to a patient with a middle ear infection such as OMTT or AOMT by inserting a delivery cannula into a tympanostomy tube in the infected ear. Such administration is preferably a one-time administration of a quantity of the composition effective to treat such middle ear infection. Compared to existing treatments of such infections using drops applied to the ear canal, the method of the present invention has a faster time to achieve the concentration of antibiotic necessary to kill the infective microbes at the infection site.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a diagram presenting a cross-section of the ear and showing typical tympanostomy tube placement;

FIG. 2 is a diagram showing administration of a pharmaceutical composition according to an embodiment of the present invention using a delivery cannula; and

FIG. 3 is a diagram of a delivery cannula and insertion indicator according to an embodiment of the present invention.

### DETAILED DESCRIPTION OF THE INVENTION

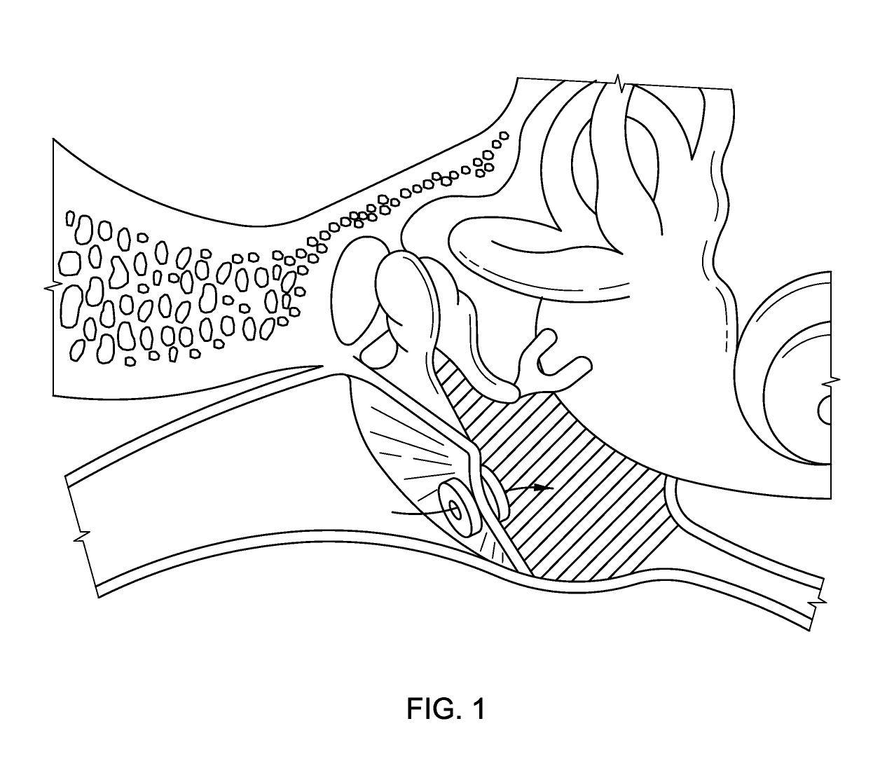

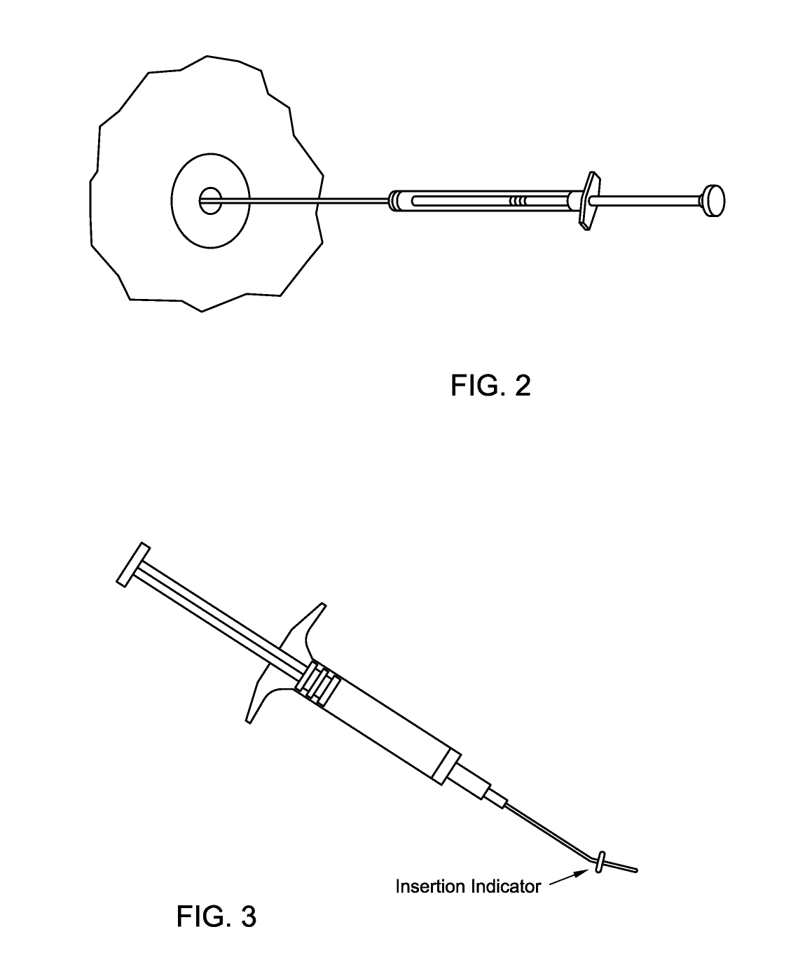

The present invention is a method for treating otic infection at the time of (such as OMTT) or following tympanostomy tube insertion (such as AOMT). FIG. 1 shows the ear anatomy and the site of typical tympanostomy tube insertion. The method comprises administering a composition comprising one or more antibiotic compounds to the site of the otic infection by instilling the composition into the tympanostomy tube using a delivery cannula, as shown in FIG. 2. As used herein, the term “delivery cannula” includes metal needles as well as other similar instruments such as hollow cannulas or catheters designed to deliver a solution or other liquid through a hollow bore. Delivery cannulas may be made of a variety of materials, but have a blunt or dull tip in preferred embodiments to avoid injuring otic tissues. For similar reasons, the delivery cannulas of the present invention are preferably made of plastic or other soft material such as polypropylene, polyethylene, polytetrafluoroethylene, or other bendable materials. Delivery cannulas may be straight (such as the one shown in FIG. 2) or may have a bent tip adapted for delivery to the otic anatomy. In certain embodiments, the tympanostomy tube is cleared of any obstructions by application of vacuum or other means known to those of skill in the art before the composition is administered.

Optionally, delivery cannulas of the present invention may have an insertion indicator attached to the outer surface of the cannula inferior to the tip. Such an insertion indicator is designed to prevent the delivery cannula from being inserted to far into the tympanostomy tube. The insertion indicator in one embodiment is a soft plastic or rubber structure encircling the outer surface of the cannula 1 to 5 mm from the tip. In a preferred embodiment, the insertion indicator is located 1 to 3 mm from the tip, and in a most preferred embodiment, the insertion indicator is located 1 to 2 mm from the tip. FIG. 3 shows one embodiment of the invention

The delivery cannulas of the present invention are sized for insertion into the bore of a tympanostomy tube. The tympanostomy tube sizes vary, but typically have an inner diameter of 1 to 1.44 mm. A preferred delivery cannula of the present invention has an outer diameter of 1 mm or less. Delivery cannulas from 19 gauge to 34 gauge are particularly preferred, and delivery cannulas of 27 to 29 gauge are most preferred.

The compositions used in the method of the present invention comprise an antibiotic compound or compounds, either alone or in combination with another therapeutic compound. In a preferred embodiment, the antibiotic compound is combined with an anti-inflammatory compound.

In a preferred embodiment, the compositions used in the method of the present invention comprise a quinolone antimicrobial or a pharmaceutically acceptable salt, derivative, enantiomer, or hydrate thereof in aqueous solution, suspension, gel, or foam. Preferred quinolone antimicrobials include those described in U.S. Pat. Nos. 4,990,517 and 5,059,597 to Petersen et al. and U.S. Pat. No. 6,716,830 to Cagel et al. (the entire contents of which are hereby incorporated by reference), and quinolones such as moxifloxacin, finafloxacin, ciprofloxacin, gatifloxacin, and ofloxacin. A particularly preferred quinolone is finafloxacin or a pharmaceutically acceptable salt, derivative, enantiomer, or hydrate thereof. Finafloxacin (8-cyano-1-cyclopropyl-6-fluoro-7-[(4aS,7aS)-hexahydro pyrrolo[3,4-b]-1,4-oxazin-6(2H)-yl]-1,4-dihydro-4-oxo-3-quinoline carboxylic acid) has the following structure:

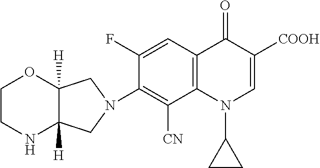 [SMILES: N#Cc1c(N2C[C@@H]3NCCO[C@H]3C2)c(F)cc2c(=O)c(C(=O)O)cn(C3CC3)c12]

A preferred quinolone salt for use in embodiments of the present invention is finafloxacin monohydrochloride. Diasteromerically and enantiomerically pure finafloxacin is also preferred for use in embodiments of the present invention. As used herein, the term finafloxacin is intended to encompass finafloxacin and its pharmaceutically acceptable salts, derivatives, enantiomers, or hydrates. The phrase “pharmaceutically acceptable” is art-recognized and refers to compositions, polymers and other materials and/or dosage forms which are suitable for use in contact with the tissues of human beings and animals without excessive toxicity, irritation, allergic response, or other problem or complication, commensurate with a reasonable benefit/risk ratio as determined by one of ordinary skill in the art. Finafloxacin and derivatives thereof can be synthesized according to the methods described in U.S. Pat. No. 6,133,260 to Matzke et al., the contents of which are herein incorporated by reference in their entirety. Finafloxacin is particularly well suited for treatment of middle ear infections, as it demonstrates high potency in the acidic conditions typically found in the environment of such infections.

It is contemplated that the concentrations of the ingredients in the compositions of the present invention can vary. In one embodiment of the present invention, a quinolone antimicrobial is present in compositions at a concentration of about 0.01% to 3.0% w/v. In a preferred embodiment, a quinolone antimicrobial is present at a concentration of 0.1% to 1.0%. In a particularly preferred embodiment, a quinolone antimicrobial is present at a concentration of about 0.3% to 0.7%. A person of ordinary skill in the art would understand that the concentrations can vary depending on the addition, substitution, and/or subtraction of ingredients in a given composition.

Compositions according to the method of the present invention are generally prepared using a buffering system that maintains the composition at a pH of about 3 to a pH of about 8. In certain embodiments, topical compositions (particularly topical ophthalmic compositions, as noted above) are preferred which have a physiological pH matching the tissue to which the composition will be applied or dispensed. For certain quinolones such as finafloxacin, the compositions generally have an acidic pH of less than 7 and generally between 4.5 and 7.5. For otic applications, an acidic pH of between 4.5 and 6 is particularly preferred. A preferred buffering system uses sodium acetate at a concentration of 0.01 to 1.0 w/v % which may increase the solubility of finafloxacin in certain finafloxacin compositions of the present invention.

In particular embodiments of the method of the present invention, a composition is administered as a one-time dose. However, the compositions of the present invention may also be formulated for administration at any frequency of administration, including once a week, once every 5 days, once every 3 days, once every 2 days, daily, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. One of ordinary skill in the art would be familiar with determining a therapeutic regimen for a specific indication that incorporates a pharmaceutically effective amount of a composition of the present invention. The phrase “pharmaceutically effective amount” is an art-recognized term, and refers to an amount of a compound that, when incorporated into a pharmaceutical composition of the present invention, produces some desired effect at a reasonable benefit/risk ratio applicable to any medical treatment. The effective amount may vary depending on such factors as the disease or infectious agent being treated, the particular composition being administered, or the severity of the disease or infection agent. The dosage volume in preferred embodiments of the present invention is typically between 50 and 1000 μL, with particularly preferred dosage volumes of 100 to 500 μL, and most preferred dosage volumes of 100 to 300 μL.

The compositions of the present invention optionally comprise one or more excipients. Excipients commonly used in pharmaceutical compositions include, but are not limited to, tonicity agents, preservatives, chelating agents, buffering agents, surfactants and antioxidants. Other excipients comprise solubilizing agents, stabilizing agents, comfort-enhancing agents, polymers, emollients, pH-adjusting agents and/or lubricants. Any of a variety of excipients may be used in compositions of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of these products.

Suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants, though nonionic surfactants are preferred, RLM 100, POE 20 cetylstearyl ethers such as Procol® CS20 and poloxamers such as Pluronic® F68. Suitable antioxidants include, but are not limited to, sulfites, ascorbates, butylated hydroxyanisole (BHA) and butylated hydroxytoluene (BHT).

The compositions set forth herein may comprise one or more preservatives. Examples of such preservatives include p-hydroxybenzoic acid ester, alkyl-mercury salts of thiosalicylic acid, such as thiomersal, phenylmercuric nitrate, phenylmercuric acetate, phenylmercuric borate, sodium perborate, sodium chlorite, benzalkonium chloride, parabens such as methylparaben or propylparaben, alcohols such as chlorobutanol, benzyl alcohol or phenyl ethanol, guanidine derivatives such as polyhexamethylene biguanide, sodium perborate, or sorbic acid. In certain embodiments, the composition may be self-preserved that no preservation agent is required.

The compositions used with a method of the present invention may also comprise one or more anti-inflammatory compounds and/or one or more analgesic compounds. The anti-inflammatory compounds utilized in the present invention are broadly classified as steroidal or non-steroidal. The preferred steroidal anti-inflammatory compounds are glucocorticoids. Glucocorticoids include dexamethasone, loteprednol, rimexolone, prednisolone, fluorometholone, hydrocortisone, fluocinolone, difluprednate, mometasone, fluticasone, beclomethasone, flunisolide, triamcinolone and budesonide. A preferred glucocorticoid is dexamethasone. Non-steroidal anti-inflammatory compounds are: prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NF.kappa.B transcription factor; or other anti-inflammatory compounds known to those skilled in the art. Analgesic compounds that may be used in embodiments of the present invention include local anesthetics such as benzocaine, tetracaine, and lidocaine, and other pain relievers such as antipyrine.

Anti-inflammatory and analgesic compounds included in the compositions of the present invention are generally at a concentration of 0.01 to 1.0 w/v %. In a preferred embodiment, anti-inflammatory or analgesic compounds included in the compositions of the present invention are at a concentration of 0.05 to 0.3 w/v %. In a particularly preferred embodiment, anti-inflammatory or analgesic compounds included in the compositions of the present invention are at a concentration of 0.1 to 0.3 w/v %.

Another embodiment of the present invention is a device for use as a single use, disposable, sterile kit for the treatment of middle ear infection following tympanostomy tube placement. The device comprises a prefilled sterile piston syringe containing from 10 to 1000 microliters of a composition comprising an antibiotic. The syringe includes a delivery cannula with a tip sized for insertion into a tympanostomy tube, and a removable cap on the tip to prevent escape of the composition and to prevent movement of the syringe piston. The kit further includes a disposable pouch for enclosing the syringe.

Embodiments of the present invention are suitable for treating a variety of otic infections, and are well suited for treating acute otitis media with tympanostomy tubes (AOMT), particularly in the context of Gram-positive and Gram-negative bacterial infections.

The following examples are presented to further illustrate selected embodiments of the present invention.

### EXAMPLE 1

| Ingredient | % w/v |
|---|---|
| Finafloxacin | 0.33 |
| Magnesium Chloride (hexahydrate) | 0.3 |
| Sodium Acetate (trihydrate) | 0.68 |
| Mannitol | 2.5 |
| Benzalkonium Chloride | 0.01 |
| Sodium Hydroxide/Hydrochloric Acid | q.s. to pH 5.9 |
| Purified Water | q.s. 100% |

### EXAMPLE 2

| Ingredient | % w/v |
|---|---|
| Quinolone antimicrobial | 0.6 |
| Zinc chloride | 0.3-1.0 |
| Sodium phosphate (anhydrous) | 0.3-0.7 |
| Sodium chloride | 0.7* |
| Sodium hydroxide/HCL | Adjust pH to 5.5 to 7.5 |
| Purified Water | q.s. 100% |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A method for treating otic infection following tympanostomy tube insertion comprising:
administering a composition comprising one or more antibiotic compounds to the site of said infection by instilling said composition into said tympanostomy tube.

2. A method according to claim 1, wherein said composition further comprises an anti-inflammatory compound.

3. A method according to claim 1, said composition comprising a quinolone antimicrobial or a pharmaceutically acceptable salt thereof at a concentration of 0.1 to 1.0 w/v %.

4. A method according to claim 3, said composition comprising a quinolone antimicrobial or a pharmaceutically acceptable salt thereof at a concentration of 0.3 to 0.7 w/v %.

5. A method according to claim 3 wherein said quinolone antimicrobial is selected from the group consisting of:
finafloxacin, ciprofloxacin, gatifloxacin, moxifloxacin and ofloxacin.

6. A method according to claim 3 wherein said quinolone antimicrobial is finafloxacin.

7. A method according to claim 1, said composition further comprising an anti-inflammatory compound.

8. A method according to claim 7, wherein said anti-inflammatory compound is a steroid.

9. A method according to claim 8, wherein said anti-inflammatory compound is selected from the group consisting of:
dexamethasone, loteprednol, rimexolone, prednisolone, fluorometholone, hydrocortisone, mometasone, fluticasone, beclomethasone, flunisolide, triamcinolone and budesonide.

10. A method according to claim 1, said composition having a pH of 4.5 to 7.5.

11. A method according to claim 1, said composition having a pH of 5.0 to 6.0.

12. A method according to claim 1, wherein said administering is performed using a delivery cannula.

13. A method according to claim 9, wherein said typanostomy tube is cleared before said administering.

14. A method according to claim 1, wherein said infection is otitis media at the time of tube placement (OMTT) or acute otitis media with tympanostomy tubes (AOMT).

15. A device for use as a single use, disposable, sterile kit for the treatment of middle ear infection following tympanostomy tube placement, comprising:
a prefilled sterile piston syringe containing from 10 to 1000 microliters of a composition comprising an antibiotic, said syringe including a delivery cannula with a tip sized for insertion into a tympanostomy tube, a cap on said tip removably mounted to selectively prevent escape of said composition from said syringe and prevent movement of said piston while said cap is on said tip, said kit further including a disposable pouch for enclosing said filled syringe.
